(19) United States
(12) Patent Application Publication
(10) Pub. No.: US 2009/0285866 A1
(43) Pub. Date: Nov. 19, 2009

(54) Self-healing polymer compositions

(76) Inventors: Isabelle R. Afriat, Plainview, NY (US); Milan F. Sojka, Corman, NY (US); Phillip Cummins, Livingston, NJ (US); Paolo U. Giacomoni, Commack, NY (US)

Correspondence Address: THE ESTEE LAUDER COS, INC, 155 PINELAWN ROAD, STE 345 S, MELVILLE, NY, 11747, US

(21) Appl. No.: 12/119,623

(22) Filed: May 13, 2008

(51) Int. Cl. A61K 8/02; A61Q 1/10; A61Q 17/04

(52) U.S. Cl. 424/401; 424/70.7; 424/59

(57) ABSTRACT

A composition including a film-former and a porous polymeric release system comprising a solvent for the film-former is provided. Also provided is a method of prolonging the physical stability of a film-former on the skin by applying to the skin a self-healing composition containing the film-former and a porous polymeric release system comprising a solvent for the film-former.

### BACKGROUND OF THE INVENTION

The present invention relates to a composition and method for treating the skin, including the lips, and the nails, and more particularly, to film-former-containing skin-care compositions, including skin-tightening compositions, which when topically applied to the skin, demonstrate improved wear and provide a skin-tightening effect with improved comfort and performance.

The process of skin ageing is typically accompanied by a gradual modification of the skin structure and functions. The principal clinical signs of skin ageing are the appearance of wrinkles and fine lines, which increase with age. In the consumer's eternal search for the fountain of youth, attention has been directed to developing compositions which will eliminate, shrink, or mask fine lines and wrinkles in the skin. Many products along these lines are known in the art. As an example, cosmetics have long included humectants, for example, hyaluronic acid, which absorb or retain moisture. These cosmetics may have the effect of temporarily smoothing out wrinkles because the skin swells where the humectant is applied. It also is known to correct these signs of ageing by using cosmetic or dermatological compositions that contains active ingredients such as alpha-hydroxy acids, beta-hydroxy acids and retinoids. The use of alpha-hydroxy acids is known from, for example, U.S. Pat. Nos. 5,091,171; 5,385,938 and 5,422,370. These active ingredients are believed to act on the wrinkles by eliminating the dead cells and by accelerating the process of cell renewal. However, the visible effect of these compositions occurs only after a certain period of application, which may range from a few days to several weeks, and the active ingredients are often irritating to skin.

Other products which have been developed to combat lines and wrinkles are those which tighten the skin upon application of the product. The tightening agents may be polymers of natural or synthetic origin. Preferred natural polymers with tightening effect include the polymers of plant origin, egg proteins, latexes of natural origin, polysaccharides, and combinations thereof. Typical examples include egg albumin and silk proteins. The polymers are capable of forming a film which causes the retraction of the stratum corneum, the superficial horny layer of the epidermis. These tightening agents, which are also known as film formers, are materials which, upon drying produce a continuous film on skin, hair or nails. The film-forming polymer component forms a continuous layer on the skin which retracts upon drying to tighten the skin. Optimally, the film does not flake off or form cracks after simple flexing of the substrate to which it is adhered. These films are used in cosmetics for diverse purposes, e.g. in forming facial masks, make-up films, hair-holding products and nail lacquers. By tightening the superficial layer of the skin, skin care compositions containing the film-forming component are capable of making the skin smooth by reducing the number and depth of the wrinkles and fine lines and of making fatigue marks disappear, this being achieved instantly. In a hair care composition, the film formed on hair makes it possible to impart more hold and softness to the hair. In a nail lacquer, a hard and shiny film can be formed which adheres thoroughly to the nails, as described in U.S. Pat. No. 6,113,930.

In the context of a skin care composition, tightening polymeric systems, though very effective and rapid, sometimes cause a feeling of discomfort in some users, in particular those having fragile skin, since the film formed on the skin may be experienced as too rigid or not sufficiently flexible. In fact, the skin in the eye area is typically thin and fragile for most consumers. Nevertheless, such formulations for the eyes are highly desired by consumers. U.S. Pat. No. 6,284,233 addresses this problem by combining highly branched or dendritic polyesters with terminal hydroxyl groups which, by themselves, have no epidermis-tightening power, with known polymeric tightening systems, to reinforce the tightening effect of the latter while using smaller quantities of tightening polymers.

A further undesirable property of skin-tightening compositions is that the films formed on the skin tend to whiten over time, as they dry. Additionally, while the films need to be thin to provide the desired tightening effect while avoiding the feeling of discomfort, once applied to the skin, the thin films tend to crack when dried or after repeated deformations of the skin induced, for example, by facial motion. As solvent evaporates and the film dries, it spontaneously breaks down into holes and droplets due to the action of weak forces which act between molecules of the thin film. Although these intermolecular forces are weak, they may gather enough collective energy, when a large number of molecules is involved, to rupture the film. These defects allow mechanical and sometimes molecular separation of the film from the substrate to occur. Damage can be induced as well by fatigue; that is, the formation of microvoids incurred by mechanical stress over time through repeated usage. The microvoids enlarge and coalesce to form microcracks in the material. This type of damage can occur to a skin tightener by the natural facial expressions such as smiling, laughing, and so forth (internal forces). Damage may also be caused by a scratch on a nail lacquer, for example (external forces).

The use of plasticizers is one approach to dealing with the disadvantages of using film-formers in cosmetic compositions. Plasticizers have typically been added to such compositions to avoid the brittleness, cracking and whitening caused by the film formers, and thereby improve the wear and appearance of these cosmetic compositions. The use of plasticizers reduces evaporation rate and imparts flexibility and toughness to the polymers. Water, sometimes used in combination with hygroscopic materials, is the common plasticizer for natural polymers, e.g., proteins. A variety or organic substances has been found useful for plasticizing synthetic polymers e.g. alkyl citrates, phthalate-based, camphor, and epoxidized vegetable oils. Plasticizers have typically been used in wrinkle-masking, film-former-containing cosmetic compositions, such as described in U.S. Pat. No. 4,965,071, to improve the overall flexibility of the composition, promote better adherence of the film to skin, and allow the film to adapt to dimensional changes associated with changing skin configuration. The plasticizers allow the flexibility of the film to be adjusted without reducing its strength or its physical force. Water-soluble polymeric films are plasticized with agents such as glycols and polyols. Suitable plasticizers include glycerin, propylene glycol, hexylene glycol and the like.

One disadvantage of using plasticizers in skin-tightening compositions is that although they may minimize the observed cracking and whitening effects, they also reduce the tightening effect consumers expect from the use of the product.

Another approach to dealing with the drying and cracking of film-formers is the “controlled release” system. The critical properties of controlled release systems are that the active ingredient must be entrapped in a certain matrix or reservoir and the vehicle must be designed appropriately to support the functionality of the delivery system.

Controlled release systems which are also “self-repairing” or “self-healing” systems for flexible polymeric materials are known. Composite materials are used in civil engineering, aerospace and defense-related projects, offshore oil exploration, electronics and biomedicine. The most common route to failure is fatigue, i.e. the formation of microvoids incurred by mechanical stress through repeated usage. The microvoids enlarge and coalesce to form microcracks in the material; in turn the microcracks can grow to catastrophic proportion and cause total failure of the product. The design of a self-repairing composite requires that the system not adversely affect the material's overall properties or performance. The system must be able to sense damage and be able to react to that damage and initiate healing. Finally, the system must restore the material's original properties. As one example, a self-healing system is established by incorporating a microencapsulated healing agent and an embedded chemical catalyst within a polymer matrix. A microencapsulated healing agent is embedded in a structural composite matrix containing a catalyst capable of polymerizing the healing agent. When cracks form in the matrix as a result of damage, the crack ruptures the microcapsules, releasing the healing agent, e.g. polymerizable monomers, epoxies, sealants, into the crack through capillary action. The healing agent contacts the catalyst, triggering polymerization that bonds the crack faces closed. U.S. Pat. Nos. 5,561,173; 660,624; 6,261,360; and U.S. published patent application 20060169180, describe such self-repairing systems.

In the context of cosmetic applications, the controlled-release delivery systems currently used in cosmetic and personal-care products fall into three main groups: microcapsules, liposomes and porous polymeric systems (e.g., microspheres). The microencapsulation technique enables liquids or solids to be wrapped in a membrane of a synthetic or natural polymer, or lipid that isolates and protects the active agent from the environment. Microcapsule size can vary from 0.5 micrometers to 2,000 micrometers. The encapsulated product, typically an active, is released either when the membrane is broken, e.g. punctured, or by slow and progressive diffusion through the membrane.

A liposome is a small, nanometer sized spherical vesicle with a membrane composed of a phospholipid and cholesterol bilayer. Liposomes usually contain a core of aqueous solution. Liposomes form bilayers similar to those found in biomembranes, and therefore enhance the penetration of cosmetic actives into the stratum corneum. Liposomes which are used as delivery systems may encapsulate hydrophilic substances in their aqueous cores. Amphiphilic and lipophilic substances can be incorporated into the lipid bilayer. Liposomes favor the disposition of encapsulated active ingredients in the epidermis and dermis, while the permeation rate is decreased. This helps to fix active ingredients to the outermost skin layers as desired for cosmetic products. U.S. Pat. No. 5,876,736 describes film-former-containing makeup compositions containing a polysaccharide-derived film forming component and a liposome-encapsulated or phospholipid-encapsulated moisturizer and/or re-hydrating ingredients, for example, panthenol, a vitamin A or a vitamin E derivative, a natural oil, a medium chain fatty acid ester of glycerol, mineral water, silicones, and silicone derivatives.

As an example of a porous polymeric delivery system, microspheres may be solid or hollow and may encapsulate a pharmacologically active substance or a cosmetic agent. Microspheres are defined as substantially spherical particles. U.S. Pat. No. 6,969,531 describes biocompatible functionalized hyaluronic acid microspheres comprising dihdrazide pendant groups. The derivatized hyaluronan microspheres have useful pharmacological and cosmetic applications as delivery vehicles for active pharmacological and cosmetic agents. U.S. Pat. No. 5,288,502 describes a delivery system for a protein, peptide, or drug with biodegradable multi-phase microspheres, said microspheres comprising a protein, peptide, or drug contained within a fixed oil and an essentially water-insoluble, biodegradable polymeric matrix comprised of polylactic acid or polylactic glycolic acid wherein the polymeric matrix surrounds the fixed oil of the microsphere and wherein the fixed oil contains the protein, peptide, or drug. Co-pending U.S. patent application Ser. No. 11/534,076 describes cosmetic compositions containing thermoplastic hollow microspheres with entrapped skin benefit agents.

To the Inventors' knowledge, however, these controlled release systems have not heretofore been applied to cosmetic compositions which are also “self-repairing” or “self-healing”. It now has been discovered, surprisingly, that a continuous release of a solvent of the film-forming polymer (flexible film) or an immediate release of a solution or a dispersion of the film-forming component in the solvent for the film-former, which dissolves and reanneals the film (hard film), can improve the wear of the composition by maintaining the integrity of the film. Moreover, since the film does not dry out to the extent that whitening and cracking occur, the compositions of the invention need not include a plasticizer to maintain the suppleness of the film.

There is therefore a need to improve the wear, comfort and performance of film-former-containing cosmetic compositions without sacrificing the desired tightening efficacy of the product. The Applicants have surprisingly discovered that a cosmetic composition including a film-former and a porous controlled release system comprising a solvent for the film-former maintains its physical stability, e.g. integrity and flexibility, and demonstrates improved properties.

### SUMMARY OF THE INVENTION

The invention relates to cosmetic compositions containing a film-former and a porous polymeric release system comprising a solvent for the film-former. The film integrity is maintained by the release of a solvent for the film-former from the porous polymeric release system. The invention also concerns methods of prolonging the physical stability of a film on the skin by applying to the skin a self-healing composition comprising a film-former and a porous polymeric release system comprising a solvent for the film-former.

### BRIEF DESCRIPTION OF THE DRAWINGS

FIG. 1 is a graph representing the effect of skin surface friction/suppleness after topical treatment of a composition according to the present invention comprising a film former and a polymeric release system as compared with the same composition without the polymeric release system.

FIG. 2 is a graph illustrating the degree of comfort observed by panelists after the application of a product containing a composition according to the present invention comprising a film former and a polymeric release system as compared with the same composition without the polymeric release system.

### DETAILED DESCRIPTION OF THE PREFERRED EMBODIMENTS

In accordance with the present invention, new and improved “self healing” cosmetic compositions comprise a film-former and a porous polymeric release system comprising a solvent of the film-former. In the context of the present invention, “self-healing” describes the ability of the film to return to its original shape or appearance after being deformed, such as by internal forces (e.g. facial expressions) or by external forces (e.g. scratching).

The film-forming polymers useful in the present invention include, but are not limited to, polyacrylates and derivatives thereof, silicone polymers and derivatives thereof, silicone elastomers and derivatives thereof, silicone resins and derivatives thereof, silicone gums and derivatives thereof, polyethylenes and derivatives thereof, polyurethanes and derivatives thereof, polysaccharides and derivatives thereof, hydrocarbonated polymers and derivatives thereof, proteins and derivatives thereof, and nitrocellulose. A preferred film-forming component is a polysaccharide or a derivative thereof, for example, hyaluronic acid. Hyaluronic acid is the skin's agent for hydration and support for tone and elasticity. It has molecular sponge properties as it moisturizes by forming a film on the skin enabling it to hold water, protects by stimulating desquamation of the stratum corneum thereby restoring smoothness and elasticity of tissues, lubricates, prevents moisture-loss from the skin and maintains the structural integrity of the tissues.

The film-forming component is present in the composition in an amount in the range of about 0.5 to about 20 weight percent, based on the total weight of the composition. Preferably, the film-forming component is present in the composition in an amount in the range of about 1 to about 10 weight percent, based on the total weight of the composition.

The porous polymeric controlled release delivery systems useful in the present invention may be any cosmetically acceptable solvent release system which will maintain the integrity of a film. Examples of such release systems include microcapsules, liposomes and microspheres comprising a solvent for the film former. The release system may comprise a solution or dispersion of the film former in the solvent for the film former. The preferred solvent release system comprises microspheres. The solvent is typically encapsulated within the microspheres. The microspheres are thermoplastic expandable particles having a hollow core. The basic microsphere technology is described in U.S. Pat. Nos. 3,615,972; 3,864,181; 4,006,223; 4,044,176; 4,397,799; 4,513,106; 4,722,943; and EP 056219 and 112807, the contents of each being incorporated herein by reference in their entirety. The microspheres can be made from a variety of different types of materials, for example polymers selected from acrylic acid/acrylonitrogens copolymer, e.g., acrylonitrile-methacrylonitrile-methyl methacrylate copolymer; allyl methacrylate dimethacrylate crosspolymer; lauryl methacrylate/glycol dimethacrylate crosspolymer; methylmethacrylate/glycol dimethacrylate crosspolymer; and nylon. Such materials are selected so as to impart the microspheres with a desirable absorption capacity for the solvent, as well as for flexibility and resistance to shear stress. The microspheres useful in the invention have a particle diameter of from about 1 to about 200 micrometers, preferably from about 5 to about 100 micrometers. However, it will be understood that the preferred particle size will depend on the microsphere loading capacity and on the desired sensorial aspect of the final product.

Microspheres of this type are commercially available, for example, Polytrap® (allyl methacrylate/glycol dimethacrylate crosspolymer) from Dow Corning; Microsponge® (methyl methacrylate/glycol dimethacrylate crosspolymer) from Cardinal Health Topical Tech, nylon microspheres from Elf Atochem, and Poly-Pore® acrylate copolymers from Health and Beauty Solution of Amcol International Company. Microspheres made from acrylonitrile-methacrylonitrile-methyl methacrylate copolymer, and known by the tradename Expancel® DE, sold by Akso Nobel, Arnhem, Amsterdam, are particularly preferred for their high solvent loading capacity (i.e. absorption and delivery capability). Examples of these microspheres include Expancel® 091 DE 40 d30, Expancel® 091 DE 80 d30, Expancel® 461 DE 20 d70, Expancel® 461 DE 40 d25, Expancel® 461 DE 40 d60, Expancel® 461 DE 40 d25, Expancel® 551 DE 40d42, Expancel® 551 DE 20 d60, Expancel® 551 DE 80 d42 and Expancel® 051 DE 40 d60. The first three digits in the particle name identify the ratios of the component monomers. For example, “091” has the monomer composition: 0:9:1 ratio of polyvinylidene chloride, poly-acrylonitrile, and polymethacrylonitrile, respectively. “551” is 55:1 ratio of polyvinylidene chloride to polyvinylidene chloride to polyacrylonitrile. “461” is 46:1 ratio of polyvinylidene chloride to polyacrylonitrile. “051” has the monomer composition: 0:5:1 ratio of polyvinylidene chloride, polyacrylonitrile, and polymethacrylonitrile. The “DE” designation identifies the material as “dry expanded”, and the “40” or “20” following it defines the median particle diameter in micrometers. The final designation “d_indicates the particles' true density in kg³/m.

Microspheres, as described above, as manufactured, have a hollow core that is occupied by a gas, which is typically a hydrocarbon gas such as butane or pentane, or air. In the compositions of the present invention, at least a portion of that gas is replaced by a solvent for the film-forming component or by a solution or dispersion of the solvent. The modification of the microspheres so as to allow them to take in the solvent is achieved by polymer and solvent interaction. Expancel® is able to entrap/load fluid in an amount of up to 18 times its weight; that is, up to 18 grams of fluid per one gram of microspheres. Loading the microspheres is achieved by freely mixing the microspheres gently with a solvent in any type of mixing device.

The polymeric controlled release delivery system is present in the composition in an amount in the range of from about 0.1 to about 10 weight percent, preferably from about 0.5 to about 5.0 weight percent, based on the total weight of the composition.

Solvents useful in the compositions of the present invention include any cosmetically acceptable solvent for the film-former which is protective of the integrity of the film; that is, a solvent which is capable of repairing breaches, e.g. microcracks or holes in the film. If the polymer is oil-soluble, the solvent will be an oil, for example, C₁₀ or greater hydrocarbons, for example, C₁₀-C₂₅ hydrocarbons, such as isododecane; esters; silicones; fatty alcohols; fatty acids; or waxes. Most preferred are C₁₀-C₂₅ hydrocarbons. If the polymer is water-soluble, useful solvents include water, alcohols, diols and ketones. The preferred solvent will be water.

The cosmetic effect of the compositions of the invention is based on maintaining the integrity of the film-former component of the composition by release of the solvent of the film-former from the porous polymeric release system, e.g., microspheres. After the application of the composition to the skin or the hair, as the film-former component dries on the skin, nail or hair surface, pressure is placed on the microspheres causing them to release the solvent. As the film rehydrates, the solvent release slows down. In accordance with the present invention, the microspheres release the solvent for the film-former to maintain the suppleness or flexibility of the film (e.g., a hyaluronic acid-based film).

The porous polymeric release system may include any ingredient that is soluble or dispersible in the solvent for the film-former, and which would provide a benefit to the product, including pigments for longer-lasting color, esters for longer-lasting spreadability and wear, and glossy silicone polymers to maintain a continuous shine, for example in lip products. The compositions of the invention may further include any cosmetic ingredient or active suitable for use in cosmetic formulations, including, but not limited to, moisturizers, emollients, anti-Irritating, anti-Inflammatory, and/or healing agents, humectants, vitamins, sunscreens, antioxidants, fillers, oils or waxes, fragrances, surfactants, thickening agents, and emulsifiers, anti-oxidants, preservatives, light-reflective particles, and so forth. Although not required to obtain the benefits described herein, the compositions may also include plasticizers. If used, plasticizers may be present in an amount in the range of about 0.1 to 50% by weight of the solution to be loaded.

The mechanism of release or delivery of cosmetic ingredients from the microspheres will generally depend upon the material entrapped. Fragrances, volatile silicones, alcohol, solvents or other volatiles wick to the surface and evaporate at a rate that is dependent on temperature, vapor pressure, surface area, and amount of material adsorbed. Non-volatile ingredients such as oils or emollients also migrate to the surface of the microspheres and theoretically are released by contact with a substrate such as the skin. As the capillary metrics mechanism draws actives from the outer surfaces of the polymeric release system, more active would migrate from the inner matrix to sustain delivery. In addition, microspheres pressed during rub-out on the skin also release adsorbed actives. Another mode of delivery is through displacement. This mechanism allows the microsphere to deliver an active ingredient onto the skin and subsequently to adsorb excess oils and sebum from the skin. The mechanism works because a material, for example, glycerin, whose surface energy is higher than that of the polymer, when released from the microsphere, is easily replaced by materials of greater affinity for the polymer. The surface energy of the polymer has a much greater affinity for skin oils than for the glycerin, for example.

Uses contemplated for the self-healing compositions according to the present invention include any cosmetic composition including a film-forming component, where it is desired to prevent drying, shrinking, and cracking of the film thus-produced, and thus maintain the integrity of the film, including, but not limited to skin-tightening compositions, moisturizing compositions, lipstick, mascara, nail lacquer and hair conditioner. In particular, lipstick and skin-tightening compositions remain flexible and comfortable and demonstrate improved wear. Hair conditioning compositions formulated in accordance with the present invention, for example, including a silicone polymer, coat and smooth the hair and add shine. The compositions of the invention may be utilized in any type of topical formulation, including aqueous, anhydrous, or water and oil-containing compositions such as emulsions. They may be incorporated into any product form, such as creams, lotions, serums and cream gels, milks, ointments, pastes, mousses, sprays, sticks, dispersions, and suspensions. Techniques for formulation of various types of vehicles are well known to those skilled in the art, and may be found, for example, in Chemistry and Technology of the Cosmetics and Toiletries Industry, Williams and Schmitt, eds, Blackie Academic and Professional, Second Edition, 1996, Harry's Cosmecology, Eighth edition, M. Reiger, ed. (2000), and Remington: The Science and Practice of Pharmacy, Twentieth edition, A. Gennaro, ed., (2003), the contents of each of these being incorporated herein by reference.

A further understanding of the invention may be obtained by reference to the following examples which are provided herein for purposes of illustration only and which are not intended to be limiting.

### EXAMPLES

### Example 1

### Improved Performance of a Hyaluronic Acid Film—In Vivo Tests

In this study, biophysical techniques are employed to evaluate the effect of skin surface friction/suppleness and moisturization with topical treatment of a composition comprising a healing polymer according to the present invention containing Expancel® loaded with water (product 1) as compared with the same composition without the Expancel® (product 2). The Gas Bearing Electrodynamometer (GBE) method is used to measure skin suppleness and the Dermal Phase Meter (DPM) method is used to measure skin moisturization.

TABLE 1
Product 1

| MATERIAL | WEIGHT PERCENT |
|---|---|
| PHASE 1 | |
| purified water | 1.962 |
| sodium hyaluronate | 0.020 |
| phenoxyethanol | 0.018 |
| PHASE 2 | |
| polyacrylamide/C13-14 | 4.360 |
| isoparaffin/laureth-7 | |
| purified water | 82.840 |
| PHASE 3 | |
| phenoxyethanol | 0.800 |
| PHASE 4 | |
| purified water | 9.500 |
| acrylic acid/acrylonitrogens | 0.500 |
| copolymer (Expancel 091 | |
| DE40 D30) | |
| TOTAL | 100.000 |

TABLE 2
Product 2

| MATERIAL | WEIGHT PERCENT |
|---|---|
| PHASE 1 | |
| purified water | 1.962 |
| sodium hyaluronate | 0.200 |
| phenoxyethanol | 0.018 |
| PHASE 2 | |
| polyacrylamide/C₁₃-C₁₄ | 4.400 |
| isoparaffin/laureth-7 | |
| purified water | 83.600 |
| PHASE 3 | |
| phenoxyethanol | 0.800 |
| PHASE 4 | |
| purified water | 9.200 |
| TOTAL | 100.00 |

All phases are prepared at room temperature. Product 1 is prepared as follows. Phase 1 is prepared separately by mixing the water and the phenoxyethanol with a helix, and then slowly adding the sodium hyaluronate and mixing until complete homogeneity of the blend is achieved. Phase 2 is prepared by adding the polyacrylamide/C₁₃₋₁₄ isoparaffin/laureth-7 to the water and mixing with a helix. Phase 4 is prepared by adding water to the acrylic acid/acrylonitrogens copolymer (Expancel 091 DE 40 D30) with a spatula. Phases 3 and 4 are then added to Phase 2. Phase 1 is added to the blend using a helix until the composition achieves homogeneity. Product 2 is prepared in the same manner as is Product 1 with the exception that Phase 4 is only water.

Experimental design: Eight healthy women are informed in detail as to the purpose and requirements of the study and are admitted if they meet the following inclusion and none of the exclusion criteria. Volunteers may not demonstrate any kind of skin disease (infection, inflammation, sunburn, tumor, and the like), or have a history of hypersensitivity or allergy to cosmetic products. Woman who are pregnant or lactating also are excluded from the study. The test areas are the backs of both hands. The subjects refrain from using cosmetic products on the test areas for at least 48 hours before the test. On the day of testing subject report to the testing facility with clean, washed hands.
Test procedure: The subjects wash their hands with soap and water in the morning at the testing facility at least one hour before testing is initiated. GBE and DPM measurements are made in the following way. One to two hours after washing, baseline values are taken in triplicate. Product is then applied at 2 mg/cm² to a 16 cm² area on the backs of the hands. The product is rubbed in for a minute using a finger cot and product effect is measured immediately after product application and every hour thereafter for a total of six hours. The backs of the hands are not washed or disturbed for the duration of the test.

Assessment of skin suppleness—Skin suppleness is assessed by measuring the resistance of the skin surface to shear using the GBE (Bill Hargens, Philadelphia, Pa.). The GBE accurately measures a force acting on the skin and the mm displacement of the skin due to that force. One side of a probe is attached to the back of the hand using a double-sided adhesive disc. The other side of the probe is connected to the GBE and is gently moved back and forth over the backs of the hands by applying a small fixed force of about 5-10 g. Skin friction/suppleness is assessed by keeping the force constant and measuring the displacement of the skin (mm). The force produces an electrical signal which is proportional to the frictional resistance of the skin, and is recorded by an oscilloscope.

Assessment of skin moisturization—A DPM (NOVA Technology Corporation Model DPM 9003) is used to non-invasively measure skin surface capacitance in vivo. The capacitance reading is directly related to picoFarads of capacitance in the volume of skin that is effectively measured, and is correlated to skin water content. A uniform-pressure sensor probe is placed on the surface of the skin within an outlined area for approximately five seconds and a reading is taken.

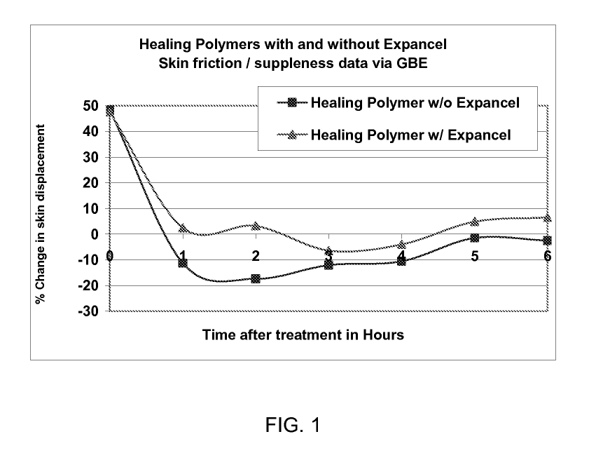

Results—Both products 1 and 2 formed films on the skin surface and demonstrated a slight decrease in skin displacement after 1 hour and throughout the six hour test period. The products did not reduce skin surface friction but rather caused slight stiffness as expected. However, product 1, containing Expancel® exhibited less surface friction and therefore higher suppleness or less stiffening/tightness than did the control product 2. The higher suppleness of product A is observed to be more pronounced in the first three hours when the film retained more of its integrity. The results are shown in FIG. 1. There is, however, no change in skin moisturization observed for both products over the six hour period.

### Example 2

### Preparation of Lip Product Compositions

Two lip product compositions are provided. As shown in Table 3, lip product A1 is prepared without the Expancel®, while lip product A2 contains Expancel® loaded with isododecane.

TABLE 3
Lip product compositions

| | WEIGHT PERCENT | |
|---|---|---|
| MATERIAL | Product A1 | Product A2 |
| trimethylsiloxysilicate polymer (DC7-4405) | 49.3 | 49.3 |
| bentone gel ISDV | 19.7 | 19.7 |
| Expancel ® 091 DE40 D30 | / | 10.0 |
| loaded with isododecane | | |
| Isododecane | 31.0 | 21.0 |
| TOTAL | 100.0 | 100.0 |

The compositions are prepared as follows. The trimethylsiloxysilicate polymer, the bentone gel and the isododecane are mixed at room temperature using a spatula and helix until a completely homogeneous mixture is formed. Expancel®, loaded with isododecane, is added to the A2 mixture using the helix.

### Example 3

### Performance of Lip Products

An expert panel of ten female subjects evaluates the two products prepared in Example 2. The panelists follow proper cleansing and product application protocols as follows:

### Cleansing Protocol Steps:

1. cleanse lips by massaging with standard cleanser to generate lather

2. rinse the lather completely

3. pat the lips dry

4. wait 30 minutes before beginning the application of lip product A1

5. repeat steps for lip product A2

### Lipstick Application Process Steps:

1. twist lipstick up ¼ inch

2. using dominant hand, apply to freshly cleansed lips, having used proper cleansing protocol

3. use standard amount of pressure (1.5 oz.)

4. Bottom lip: use six (6) strokes across from left to right

5. Top lip: use six (6) strokes from center toward left and six (6) strokes from center toward right

6. press lips together lightly but do not blot

Ten characteristics (kinesthetic and tactile) of the lip products are assessed. The kinesthetic evaluation is described as follows:

COATEDNESS- The degree to which the lipstick film has a perceived
weight on the surface of the lips. The amount of coating perceived
kinesthetically on the surface of the lips after product application
(without touching).

| | |
|---|---|
| 0 | 10 |
| Not Coated at all | Extremely Coated |

MOISTNESS- The amount of moisture or lubricated feel
perceived kinesthetically on the surface of the lips without
touching or licking the lips.

| | |
|---|---|
| 0 | 1 |
| None | Extremely moist |

TIGHTNESS - The degree of pulling or tautness perceived
kinesthetically on the lips associated with the feeling of dry lips
(without touching or licking the lips).

| | |
|---|---|
| 0 | 10 |
| None | Extremely Tight |

The tactile evaluation is described as follows:

SPREADABILITY
The tactile perception of the degree of slip of the lipstick or gloss over
the lip surface during application.

| | |
|---|---|
| 0 | 10 |
| No slip | Extreme Slip |

TACKINESS
The tactile perception of the degree to which the skin surface feels
sticky when the lips are gently pressed together and then released.
Evaluated immediately after application and at various time-points
after application.

| | |
|---|---|
| 0 | 10 |
| None | Extremely Tacky |

OILINESS
The tactile perception of a slippery, thin liquid film on the lip
surface characterized by lack of tackiness and by easy movement of
the substance across the lip surface (a very lubricated feel) and
by easy movement of the lips when gently pressed together and
moved from side to side by movement of the lower jaw. Evaluated
immediately after application and at various time-points after
application.

| | |
|---|---|
| 0 | 10 |
| Not at all | Extremely Oily |

GREASINESS
The tactile perception of a thick, dense film often accompanied by
surface drag and tackiness. The perception of moistness resulting
from solid fat rather than liquid (oil). Evaluated by gently
pressing lips together and moving lips from side to side by
movement of the lower jaw. Evaluated immediately after
application and at various time-points after application.

| | |
|---|---|
| 0 | 10 |
| Not at all | Extremely Greasy |

WAXINESS
The tactile perception of a dry coated surface with perceivable drag.
Evaluated by gently pressing lips together and moving lips from side to
side by movement of the lower jaw. Evaluated immediately after appli-
cation and at various time-points after application.

| | |
|---|---|
| 0 | 10 |
| Not at all | Extremely Waxy |

TABLE 4
lip product characteristics evaluated

| | W/O | W/ |
|---|---|---|
| | Expancel | Expancel |
| | Product | Product |
| Application | A1 | A2 |
| Spreadability | 8.4 | 8.6 |
| Greasiness | 2.4 | 2.1 |
| Oiliness | 7.6 | 7.4 |

| | | | W/ |
|---|---|---|---|
| | | W/O | Expancel |
| | | Expancel | Product |
| | | Product A2 | A1 |
| Tackiness | | | |
| Immediate | | 4.1** | 2.4** |
| ½ | hr | 5.8** | 4.4** |
| 1 | hr | 5.3** | 4.0** |
| 1½ | hr | 4.9** | 3.7** |
| 2 | hr | 4.8** | 3.7** |
| Waxiness | | | |
| Immediate | | 6.1 | 6.2 |
| ½ | hr | 6.0 | 5.9 |
| 1 | hr | 5.9 | 6.2 |
| 1½ | hr | 5.9 | 6.1 |
| 2 | hr | 5.6 | 5.4 |
| Moistness | | | |
| Baseline | | 2.0 | 2.0 |
| Immediate | | 7.2 | 6.4 |
| ½ | hr | 2.1 | 2.1 |
| 1 | hr | 1.7 | 1.6 |
| 1½ | hr | 1.6 | 1.3 |
| 2 | hr | 1.4 | 1.0 |
| Coatedness | | | |
| Immediate | | 5.2** | 4.6** |
| ½ | hr | 3.6 | 3.3 |
| 1 | hr | 3.1 | 3.2 |
| 1½ | hr | 2.8 | 2.7 |
| 2 | hr | 2.4 | 2.3 |

| | | | W/O | W/ |
|---|---|---|---|---|
| | | | Expancel | Expancel |
| | | | Product | Product |
| | | | A1 | A2 |
| Tightness | | | | |
| Baseline | | 0.1 | 7.0 | 6.9 |
| Immediate | | 0 | 2.4 | 2.2 |
| ½ | hr | 0.5 | 6.2** | 5.4** |
| 1 | hr | 1 | 6.3** | 5.4** |
| 1½ | hr | 1.5 | 6.4** | 5.4** |
| 2 | hr | 2 | 6.4** | 5.4** |
| Dry Time | | | | |
| 5 | min | 5 | 5.8** | 4.9** |
| 10 | min | 10 | 4.8 | 4.9 |
| 15 | min | 15 | 4.5 | 4.8 |
| 20 | min | 20 | 4.1 | 4.2 |
| 25 | min | 25 | 3.4 | 3.3 |
| 30 | min | 30 | 2.9 | 2.8 |
| Comfort | | | | |
| ½ | hr | 0.5 | 2.2* | 2.6* |
| 1 | hr | 1 | 1.9* | 2.4* |
| 1½ | hr | 1.5 | 1.8* | 2.3* |
| 2 | hr | 2 | 1.7* | 2.0* |

*indicates a difference between formulas at an 85% confidence level
**indicates a difference between formulas at a 95% confidence level

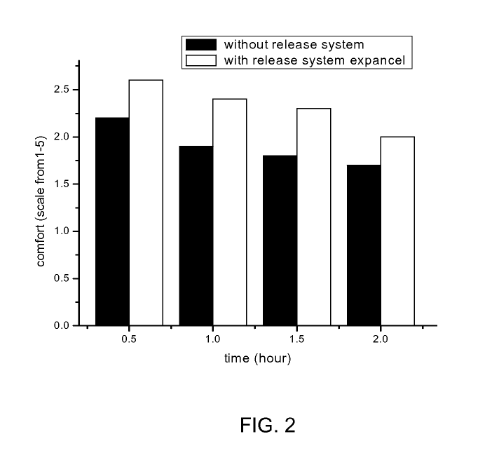

The overall comfort level of each product, primarily assessed by considering the results of the levels of tightening and moistness observed after product application, is also shown in FIG. 2. The expert panel makes the determination that, overall, lip product A2, containing the Expancel®, as compared with lip product A1, without the Expancel®, feels drier 5 minutes after application, feels less tacky on the lips after all time points, feels less coated on the lips immediately after application, and feels less tight and more comfortable on the lips from ½ hour after application through 2 hours after application. It is concluded that the continuous release of solvent (isododecane) maintains the film containing the solvent release system (Expancel® loaded with isododecane) suppler, e.g., more flexible, increasing the perception of comfort of lip product A2 on the lips.

What we claim is:

1. A composition comprising:
a film-former; and
a porous polymeric release system comprising a solvent for the film-former.

2. The composition of claim 1 wherein the porous polymeric release system comprises microspheres and wherein the solvent for the film-former is encapsulated within the microspheres.

3. The composition of claim 2 wherein the microspheres comprise a polymer selected from the group consisting of acrylic acid/acrylonitrogens copolymer, allyl methacrylate dimethacrylate crosspolymer, lauryl methacrylate/glycol di methacrylate crosspolymer, methylmethacrylate/glycol dimethacrylate crosspolymer and nylon.

4. The composition of claim 3 wherein the acrylic acid/acrylonitrogens copolymer comprises acrylonitrile-methacrylonitrile-methyl methacrylate copolymer.

5. The composition of claim 1 wherein the film-former is selected from the group consisting of polyacrylates and derivatives thereof, silicone polymers and derivatives thereof, silicone elastomers and derivatives thereof, silicone resins and derivatives thereof, silicone gums and derivatives thereof, polyethylenes and derivatives thereof, polyurethanes and derivatives thereof, polysaccharides and derivatives thereof, hydrocarbonated polymers and derivatives thereof, proteins and derivatives thereof, and nitrocellulose.

6. The composition of claim 5 wherein the film-former is a silicone polymer or a derivative thereof or a polysaccharide or a derivative thereof.

7. The composition of claim 6 wherein the film-former is hyaluronic acid or a derivative thereof.

8. The composition of claim 2 wherein the microspheres are present in the composition in an amount in the range of from about 0.1 to about 10 weight percent, based on the total weight of the composition.

9. The composition of claim 1 wherein the film former is present in the composition in an amount in the range of from about 0.5 to about 20 weight percent, based on the total weight of the composition.

10. The composition of claim 1 wherein the solvent for the film former is water or a C₁₀-C₂₅ hydrocarbon solvent.

11. The composition of claim 10 wherein the hydrocarbon solvent is isododecane.

12. The composition of claim 1 wherein the solvent for the film-former comprises a cosmetic ingredient which is a skin care active, a pigment, an ester, or a glossy polymer.

13. The composition of claim 1 which is a lip product, a mascara, a skin-tightener, a sunscreen, a moisturizer or a hair conditioner.

14. A method of prolonging the physical stability of a film-former on the skin comprising applying to the skin a self-healing composition comprising the film former and a porous polymeric release system comprising a solvent for the film former.

15. The method of claim 14 wherein the porous polymeric release system comprises microspheres and the solvent for the film-former is encapsulated within the microspheres.

16. The method of claim 15 wherein the microspheres comprise a polymer selected from acrylic acid/acrylonitrogens copolymer, allyl methacrylate dimethacrylate crosspolymer, lauryl methacrylate/glycol dimethacrylate crosspolymer, methylmethacrylate/glycol dimethacrylate crosspolymer and nylon.

17. The method of claim 16 wherein the acrylic acid/acrylonitrogens copolymer comprises acrylonitrile-methacrylonitrile-methyl methacrylate copolymer.

18. The method of claim 14 wherein the film-former is selected from the group consisting of polyacrylates and derivatives thereof, silicone polymers and derivatives thereof, silicone elastomers and derivatives thereof, silicone resins and derivatives thereof, silicone gums and derivatives thereof, polyethylenes and derivatives thereof, polyurethanes and derivatives thereof, polysaccharides and derivatives thereof, hydrocarbonated polymers and derivatives thereof, proteins and derivatives thereof, and nitrocellulose.

19. The method of claim 18 wherein the film-former is hyaluronic acid or a derivative thereof.

20. The method of claim 14 wherein the solvent for the film former is water or isododecane.
